(19) United States
(12) Patent Application Publication
(10) Pub. No.: US 2007/0134695 A1
(43) Pub. Date: Jun. 14, 2007

(54) Dengue virus detection measured by immunocytometry in a dendritic cell surrogate

(76) Inventors: Timothy Burgess, Silver Spring, MD (US); Jeffrey A. Tjaden, Eagle River, AK (US); Kevin Porter, Boyds, MD (US); Mary Marovich, Bethesda, MD (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 11/586,895

(22) Filed: Oct. 26, 2006

Related U.S. Application Data
(60) Provisional application No. 60/731,989, filed on Oct. 27, 2005.

(51) Int. Cl. C12Q 1/70; C12Q 1/68

(52) U.S. Cl. 435/6; 435/5

(57) ABSTRACT

The invention concerns a method for diagnosing dengue invention early after dengue virus infection. The method comprises binding dengue virus contained in sera from infected patients with dentritic cell-specific intracellular adhesion molecule (ICAM) 3-grabbing nonintegrin (DC-SIGN) or liver/lymph node-specific intracellular adhesion molecule (ICAM) 3-grabbing nonintegrin (L-SIGN). DC-SIGN or L-SIGN can be expressed in transfected B cells or adhered to other surfaces. Measurement of binding is by immunocytometry, microscopy or other antibody based methods.

### CROSS-REFERENCE TO RELATED APPLICATIONS

This application claims priority to U.S. Provisional application 60/731,989 filed Oct. 27, 2005.

### BACKGROUND OF INVENTION

1. Field of Invention

The inventive subject matter relates to a method of detecting of detecting dengue virus in human serum important in the detection and diagnosis of early dengue virus infection.

2. Background Art

Dengue virus, the causative agent of dengue fever and dengue hemorrhagic fever (DHF), is a virus of the genus Flavivirus, a single-stranded enveloped RNA virus with positive polarity and encoding approximately 3,400 amino acids. Dengue fever is the most common human arbovirus infection worldwide and serious public health concern accounting for estimates of 100 million infections annually (WHO 1986; Monath and Heinz 1996; Thomas, et al 2003). Dengue and DHF is found in most tropical areas including Africa, Asia, the Pacific, Australia and the Americas.

Although the virus is capable of growing in a variety of species of mosquitoes, including Aedes albopictus, Aedes polynesiensis and Aedes scutellaris, Aedes aegypti is the most efficient of the mosquito vectors because of its domestic habitat (Gubler 1988). Four antigenically distinct serotypes of dengue virus have been identified with all causing human diseases (Gubler, et al 1979; Henchal and Putnak 1990). Each of the four serotypes, although distinct, is similar enough to the others to elicit only partial cross-protection following infection (WHO 1986). Following infection, viremia is typically detected early at the onset of symptoms (Halstead 1997). Although many infections are mild, some infections result in dengue hemorrhagic fever and dengue shock syndrome, which are potentially fatal. This usually occurs in a small number of people during a second infection with a dengue virus that is different from the virus causing the first infection (Halstead 1997).

Dengue virus infection occurs following the bite of dengue virus-infected Aedes mosquitoes, which in-turn were infected by prior feeding on infected humans. Symptoms of dengue infection, including high fever, severe headache, retro-orbital pain, development of a rash, nausea, joint and muscle pain, usually start within five to six days following the bite of an infected mosquito. Symptoms of DHF also include marked sub-dermal bleeding, causing a purplish bruise, as well as bleeding from the nose and gums. The fatality rate is 6 to 30% with most deaths occurring in infants. The management of DHF is symptomatic and supportive and aimed at replacement of plasma loss (Nimmannitya 1996).

It is not possible to make an accurate diagnosis of mild or classical dengue fever based on clinical features of dengue fever since many symptoms of dengue fever resemble those of other diseases, such as chikungunya infection (Nimmannitya 1996), measles, influenza an rickettsial infections. Differential diagnosis must include malaria and other viral, bacterial and rickettsial diseases. Diagnostic methods for infection are typically based on detection of virus, viral antigens genomic sequencing and dengue-specific antibodies (Shu and Huang 2004). Dengue hemorrhagic fever (DHF) can, in some cases, however, be more accurately diagnosed from clinical symptoms, including high continuous fever for 2 to 7 days, hepatomegaly, shock and thromocytopenia.

In diagnosing dengue fever or DHF, however, it is usually necessary to base the diagnosis on results using a combination of methods. Even confirmation of dengue hemorrhagic fever requires serological analysis for assessment of seroconversion or virus isolation by infection of cell cultures or propagation in brain tissue of young mice or amplification by inoculation into mosquitoes and examination by immunofluorescence.

Antibodies to dengue virus antigens increase rapidly in patients with secondary dengue infection. A diagnostic (typically four-fold) increase in dengue antibody can often be observed during febrile illness. However, in primary dengue infection, specimens are usually acquired as late as two to three weeks after onset of disease. Confirmation of serconversion by antibody detection requires two samples, one at the beginning of the manifestation of clinical symptoms and the other 10 to 28 days later. The method of detecting antibody is usually via haemaglutination reaction (IHA) or by ELISA. For example, U.S. Pat. No. 5,824,506 to Chan, et al (1998) discloses a method for detecting antibodies to dengue in serum from infected patients using peptide antigens derived from dengue virus type-2 NS 1. However, this method may suffer from disadvantages including the fact that the selected peptides may not offer sensitive recognition of antibodies, especially early after infection.

The current “gold standard” method for detecting acute dengue infection involves virus isolation and characterization using virus culture and growing the virus in a susceptible cell line such Vero cells, an African Green monkey kidney cell line or the mosquito line C6/36, derived from the mosquito Aedes albopictus. Although the assay method is considered reliable, it suffers from serious disadvantages including relatively low sensitivity and significant time, five to ten days, before results are available.

Antibody-based assays designed to detect antigen have been described in U.S. Pat. No. 6,870,032 to Flamand, et al (2005) for detecting NS-1. This method utilizes a capture antibody directed to NS-1 using high affinity antibodies specific to the hexameric form of NS-1. This method is capable of detecting as little as 1 ng of protein/ml of serum and can be conducted within 24 hours.

Because of the importance of designing effective detection methods and therapies against dengue virus invention, considerable effort has been directed toward the mechanisms of dengue virus infection. Dendritic cells capture microorganisms and ultimately present antigens to resting T cells (Banchereau, et al 2000; Oriss, et al 2005). It this regard, it was reported that dendritic cells utilize a dendritic cell-specific intracellular adhesion molecule (ICAM) 3-grabbing nonintegrin (DC-SIGN), a C-lectin, to promote efficient infection by HIV in trans of cells that express CD4 and chemokine receptors ((I) Geijtenbeek, et al 2000). Flow cytometric analysis of the hematopoetic cells using anti-DC-SIGN antibodies demonstrated that DC-SIGN is preferentially expressed on in vitro cultured DC but not on other leukocytes, such as monocytes and peripheral blood lymphocytes ((II) Geijtenbeek, et al., 2000). Amino acid analysis of the 44-kDa protein has shown it to be identical in amino acid sequence to gpl 20-binding C-type lectin isolated from a placental cDNA library (Curtis, et al. 1992).

It was demonstrated that subsets of dendritic cells, especially those susceptible to infection with dengue virus in culture, express DC-SIGN (Marovich, et al 2001; Libraty, et al 2001). DC-SIGN also permits infection of dendritic cells by other viruses, including cytomegalovirus (CMV), Ebola virus, hepatitis C virus, HIV, Marburg virus and SARS coronavirus (Alvarez, et al, 2002; Baribaud, et al, 2002; Halary, et al, 2002; Colmenares, et al, 2002; Maeda, et al, 2003; Tailleux, et al, 2003). It was recently reported that dengue virus infection directly correlates with expression of DC-SIGN or its homologue liver/lymph node-specific specific intracellular adhesion molecule (ICAM) 3-grabbing nonintegrin (L-SIGN) (Tassaneetrithep, et al, 2003).

### BRIEF SUMMARY OF THE INVENTION

Currently available methods for the detection and diagnosis of dengue infection early after infection are relatively insensitive and require considerable time prior to receiving results. Therefore, an object of this invention is a method of detecting dengue virus infection early after infection by measuring dengue virus binding to the dendritic cell specific intracellular adhesion molecule 3-grabbing non-integrin (DC-SIGN).

Another object of the invention is a method of detecting early dengue virus infection by use of DC-SIGN and L-SIGN transfected into cell lines.

Another object of the invention is a method of detecting early dengue virus infection by detecting DC-SIGN and L-SIGN mediated dengue virus infected cells by immunocytometry.

### BRIEF DESCRIPTION OF DRAWINGS

FIG. 1. Immunocytometry scatterplots of dengue infected and mock-infected Raji transfected and Raji control (non-transfected) cell line.

FIG. 2. Dose response between dengue virus exposure and infection rate (% infection).

### DESCRIPTION OF THE PREFERRED EMBODIMENTS

Dendritic cell specific intracellular adhesion molecule 3-grabbing non-integrin (DC-SIGN) has been demonstrated to bind to dengue virus and mediate dengue virus infection of dendritic cells (Tassaneetrithep, et al, 2003). Based on this observation a method for detecting dengue infection was developed incorporating the propensity of dengue virus in patient sera to adhere to DC-SIGN. The assay method, in general, entails the specific adherence of dengue virus to DC-SIGN, expressed by DC-SIGN transfected cells or other DC-SIGN immobilizing surface and the subsequent measurement of this binding using dengue specific antibody.

The method comprises the following general steps:
a. add media, for example, RPMI-1640 supplemented with 10% fetal bovine serum (FBS), L-glutamine and penicillin/streptomycin) to wells of a 96-well round bottom culture plate;
b. add DC-SIGN in the form of cells transfected with DC-SIGN or DC-SIGN bound to other surfaces such as microbeads;
c. add serial dilutions of test sera (in replicates) or control virus to wells. For positive controls, prepare multiple dilutions of virus in media, typically from MOI=0.01 to 0.0001. Controls should include positive controls comprising virus plus media and virus plus media and serum. Additional, negative controls include media alone and media plus serum, both without virus. After addition of virus or control, incubate at 37° C. for 1 hour and wash cells with media at least twice then resuspend in media;
d. Harvest culture after 72 hours and 96 hours and monitor by indirect staining with anti-dengue antibody on slides and visualizing by light or deconvolution microscopy. Additionally, intracellular dengue is visualized by immunocytometry.

As a specific example, DC-SIGN is shown transfected into Raji B cells in order to illustrate how the inventive diagnostic method can be used in diagnosing early dengue infection. However, instead of Raji cells, DC-SIGN can be transfected into other cell lines. Alternatively, DC-SIGN can be immobilized on the surface of matrixes such as microbeads.

In this example, Raji B cells were transfected with pRc/CMV-DC-SIGN ((I) Geijtenbeek, et al 2000 ). The DC-SIGN transfected Raji B cells were grown in 75-cm² flasks with complete media comprising RPMI-1640 media containing 10% heat-inactivated fetal bovine serum, and supplemented with L-glutamine and penicillin/streptomycin. Dengue virus containing samples were prepared by growing the dengue-1, dengue-2, dengue-3 and dengue-4 virus in Vero cells. Following titration of the virus, simulated infected serum samples were made by pre-treating normal human sera with serially diluted virus.

Infection of Raji B cells was conducted by first suspending the B cells at 2×10⁶ cells/niL in complete media. To each well of a 96-well round bottom culture plate was added 30 μl (i.e. 60,000 cells). The Raji B cells were then exposed to the serially diluted dengue virus sera for 60 minutes. The procedure yields a Multiplicity of Infection (MOI) of 0.00001 to 0.001. After 60 minutes, the dengue-exposed cells were washed with complete medium and incubated at 37° C. in 5% CO₂. The study also included controls mock-infected negative control and with cells exposed to a known titer, as a positive control. Quantification of the infection virus was conducted by Vero cell plaque assay (Eckels et al 1976)

Dengue virus infection was measured by harvesting the exposed cells and controls were harvested at time points ranging from 24 to 96 hours after infection by cyto-spinning the contents of the wells onto slides which were then allowed to air-dry. The cells were then fixed with 4% paraformaldehyde. After permeabilization with 1% saponin anti-dengue virus envelope complex monoclonal antibody was applied and the slides washed multiple times followed by application of fluorochrome-conjugated secondary antibody. The slides were analyzed by either light microscopy or a deconvolution microscope.

Detection of intracellular dengue virus antigen was conducted by first fixed with 4% paraformaldehyde and permeabilized with 0.5% saponin and stained with anti-dengue virus envelope complex antibody and counterstained with a fluorescein isothiocyante (FITC) conjugated secondary antibody. The level of staining was characterized using a FACScang (Becton Dickenson, Franklin Lakes, NJ). Matches isotype control antibodies were also used in the dengue virus-exposed and mock-infected cells to assess background staining.

Table 1 illustrates the infectivity of DC-SIGN transfected Raji B cells by dengue virus serotypes 1-4. Measurements of infectivity was by immunocytometry as a function of varying virus input and time of harvest. P values for the percentage data were obtained from a Student's t test for independent samples against mock-infected cells.

TABLE 1

| | MOI^{a} | | | | | |
|---|---|---|---|---|---|---|
| | 0.0005^{b} | | 0.0001 | | 0.00001 | |
| | | P value | | P value | | P value |
| Dengue 1 | | | | | | |
| 48 hrs | 13.3% | <0.001 | 3.3% | 0.003 | 0.2% | 0.091 |
| 72 hrs | 39.0% | <0.001 | 26.3% | <0.001 | 7.3% | <0.001 |
| 96 hrs | 42.5% | <0.001 | 30.4% | 0.005 | 14.8% | <0.001 |
| Dengue 2 | | | | | | |
| 48 hrs | 12.3% | 0.682 | 2.6% | 0.11 | 0.1% | 0.102 |
| 72 hrs | 31.2% | <0.001 | 14.3% | 0.003 | 2.1% | 0.111 |
| 96 hrs | 44.3% | <0.001 | 35.8% | <0.001 | 6.9% | <0.001 |
| Dengue 3 | | | | | | |
| 48 hrs | 1.0% | 0.2 | 0.1% | 0.149 | 0.0% | — |
| 72 hrs | 8.9% | <0.001 | 2.0% | <0.001 | 0.6% | 0.001 |
| 96 hrs | 25.3% | <0.001 | 8.3% | 0.002 | 1.5% | 0.004 |
| Dengue 4 | | | | | | |
| 48 hrs | 10.8% | <0.001 | 2.2% | 0 | 0.0% | 0.22 |
| 72 hrs | 47.1% | <0.001 | 39.1% | <0.001 | 4.3% | 0.37 |
| 96 hrs | 52.1% | <0.001 | 42.2% | <0.001 | 25.7% | <0.001 |

^{a}MOI = multiplicity of infection.
^{b}MOI of 0.001 corresponds to an input of 60 plaque forming units (PFU).

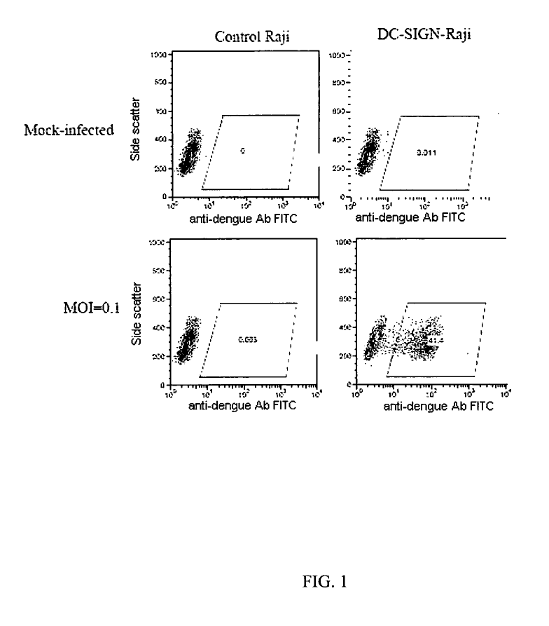

FIG. 1 illustrates infection of the normally dengue virus-resistant Raji B cell line that has been transfected with DC-SIGN by flowcytometry. The flowcytometry scatterplots show the Raji control cell line and Raji cell line transfected with DC-SIGN gated on anti-dengue virus (anti-preM-antigen) expression. Both cell lines were exposed to dengue serotypes 1-4 at MOI=0.1 or mock-infected and harvested for intracellular preM-antigen staining after 24 hours. Raji control cells show a background fluorescence of 0.0%, while 0.011% of mock-infected and 41.4% of DC-SIGN-Raji cells are positive for intracellular preM-antigen. The X-axis illustrates relative fluorescence using anti-dengue-FITC conjugated antibody. From these results, discrimination of pre-M-antigen-positive cells is readily demonstrated.

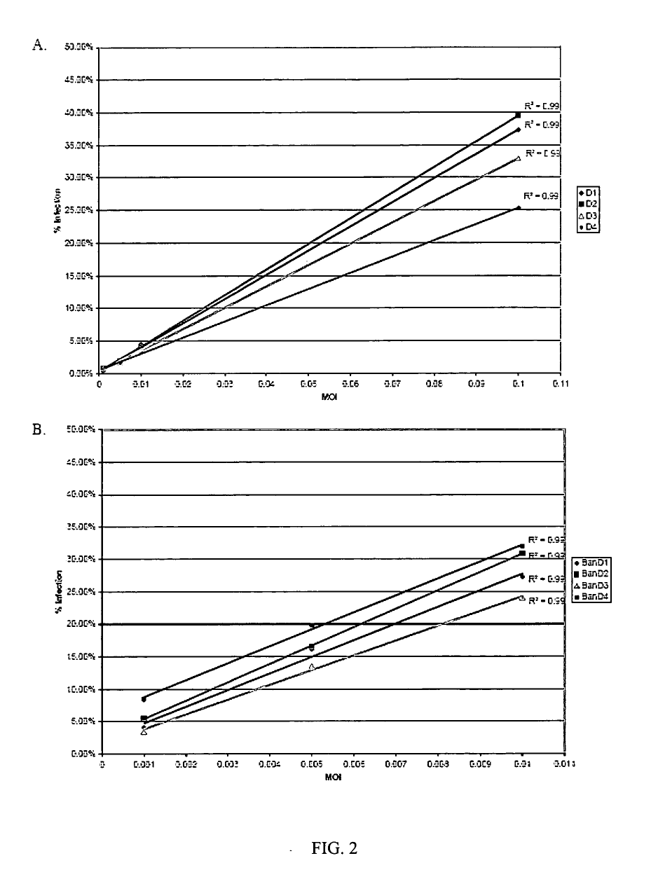

FIG. 2 illustrates a linear dose response between virus input and the number of preM-antigen-positive cells. Panel (A) shows DC-SIGN transfected cells that were infected with an increasing MOI of stock dengue virus serotypes 1-4 (D1 WP74, D2 16803, D3 CH53489, D4 Carib). Panel (B) shows low passage wild-type dengue viruses, isolated from infected patients from Bandung, Indonesia, starting with an MOI of 0.001 and incubated for 24 hours. Regression analysis of MOI data shows a clear linear relationship.

### REFERENCES

1. Alvarez, C. P., F. Lasala, J. Carrillo, O. Muniz, A. L. Corbi, and R. Delgado. 2002. C-type lectins DC-SIGN and L-SIGN mediate cellular entry by Ebola virus in cis and in trans. J. Viro. 76:6841-6844.
2. Baribaud, F., S. Pohlmann, G. Leslie, F. Mortari, and R. W. Doms. 2002. Quantitative expression and virus transmission analysis of DC-SIGN on moncyte-derived dendritic cells. J. Virol. 76:9135-9142.
3. Colmenares, M., A. Puig-Kroger, O. Muniz Pello, A. L. Corbi and L. Rivas. 2002. Dendritic-cell specific ICAM-3 grabbing nonintegrin (DC-SIGN, CD209), a C-type surface lectin in human dendritic cells, is a receptor for Leishmania amastigotes. J. Biol. Chem. 277:36766-36769.
4. Chan, L. and M. Guan. U.S. Pat. No. 5,824,506, October 1998.
5. Curtis, B. M., S. Schamowske, and A. J. Watson. 1992. Sequence and expression of a membrane-associated C-type lectin that exhibits CD4-independent binding of human immunodeficiency virus envelope glycoprotein gp120. Proc. Natl. Acad. Sci. USA 89: 8356-8360.
6. Eckels, K. H., W. E. Brandt, V. R. Harrison, J. M. McCown, and P. K. Russell. 1976. Isolation of a temperature-sensitive dengue-2 virus under conditions suitable for vaccine development. Infect. Immun. 14:1221-1227.
7. Flamand, M., F. Megret, S. Alcon, A. Talarnin, P. Despres, V. Deubel. U.S. Pat. No. 6,870,032, March 2005.
8. (I) Geijtenbeek, T. B., D. S. Kwon, R. Torensma, S. J. van Vliet, G. C. van Duijnhoven, J. Middel, I. L. Cornelissen, H. S. Nottet, V. N. KewalRamani, D. R. Littman, C. G. Fifdor, and Y. van Kooyk. 2000. DC-SIGN, a dendritic cell-specific HIV-1-binding protein that enhances trans-infection of T cells. Cell. 100:587-597.
9. (II) Geijtenbeek, T. B., R. Torensma, S. J. van Vliet, G. C. van Duijnhoven, G. J. Adema, Y. van Kooyk and C. G. Figdor. 2000. Identification of DC-SIGN, a novel dendritic cell-specific ICAM-3 receptor that support primary immune responses. Cell 100:575-585.
10. Geijtenbeek, T. B. H., S. J. van Vliet, E. A. Koppel, M. Sanchez-Hernandez, C. M. J. E. Vandenbroucke-Grauls, B. Appelmelk and Y. Van Kooyk. 2003. Mycobacteria target DC-SIGN to suppress dendritic cell function. J. Exp. Med. 197:7-17.
11. Gubler, D. J., S. Nalim, R. Tan, H. Saipan, and J. Sulianti Saroso. 1979. Vqariation in susceptibility to oral infection with dengue viruses among geographic strains of Aedes aegypti. Am. J. Trop. Med. Hyg. 28:1045-1052.
12. Gubler, D. I. 1988. Dengue. In The Arboviruses: Epidemiology and Ecology. T. P. Monath (ed.), CRC Press (Boca Raton), p223-260.
13. Halstead, S. B. 1997. Epidemiology of dengue and dengue hemorrhagic fever. In Dengue and Dengue Hemorrhagic Fever. D. J. Gubler and G. Kuno, editors. Cab international, London. 23-44.
14. Henchal, E. A. and J. R. Putnak. 1990. The dengue viruses. Clin. Microbiol. Rev. 3:376-396.
15. Libraty, D. H., S. Pichyangkul, C. Ajariyakhajorn, T. P. Endy, and F. A. Ennis. 2001. Human dendritic cells are activated by dengue virus infection: enhancement by gamma interferon and implications for disease pathogenesis. J. Virol. 75:3501-3508.
16. Maeda, N., J. Nigou, J. L. Herrmann, M. Jackson, A. Amara, P. H. Lagrange, G. Puzo, B. Gicquel and O. Neyrolles. 2003. The cell surface receptor DC-SIGN discriminates between Mycobacterium species through selective recognition of the mannose caps on lipoarabinomannan. J. Biol. Chem. 278:5513-5516.
17. Marovich, M., G. Grouard-Vogel, M. Louder, M. Eller, W. Sun, S. J. Wu, R. Putvatana, G. Murphy, B. Tassaneetrithep, T Burgess, et al. 2001. Human dendritic cells as targets of dengue virus infection. J. Investig. Dermatol. Symp. Proc. 6:219-224.
18. Monath, T. P., and F. X. Heinz. 1996. Flaviviruses. In Fields Virology. B. N. Fields, D. M. Knipe and P. M. Howley, (eds.) Lippincott-Raven, Philadelphia. 961-1034.
19. Nimmannitya, S. 1996. Dengue and dengue haemorrhagic fever. In Manson's Tropical Diseases. G. C. Cook (eds.) W. B. Saunders Company, Ltd (London). 721-729.
20. Shu, P. Y., and J. H. Huang. 2004. Current advances in dengue diagnosis. Clin. Diagn. Lab. Immunol., 11(4):642-650.
21. Tailleux, L., O. Schwartz, J.-L. Herrmann, E. Pivert, M. Jaackson, A. Amara, L. Legres, D. Dreher, L. P. Nicod, J. C. Gluckman, et al. 2003. DC-SIGN is the major mycobacterium tuberculosis receptor on human dendritic cells. J. Exp. Med. 197:121-127.
22. Thomas, S. J., D. Strickman, and D. W. Vaughn. 2003. Dengue epidemiology: virus epidemiology, ecology and emergence. Adv. Virus Res. 61:235-289.
23. World Health Organization. Dengue Hemorrhagic Fever: Diagnosis, Treatment and Control. Geneva: WHO, 1986.

Having described the invention, one of skill in the art will appreciate in the appended claims that many modifications and variations of the present invention are possible in light of the above teachings. It is therefore, to be understood that, within the scope of the appended claims, the invention may be practiced otherwise than as specifically described.

What is claimed is:

1. A dengue virus detection method comprising:
a. exposing patient sera containing dengue virus to Dendritic cell-specific intracellular adhesion molecule (ICAM) 3-grabbing nonintegrin (DC-SIGN) or liver/lymph node-specific intracellular adhesion molecule (ICAM) 3-grabbing nonintegrin (L-SIGN);
b. measuring dengue virus bound to said DC-SIGN or L-SIGN.

2. The method of claim 1, wherein said DC-SIGN or L-SIGN is expressed on DC-SIGN or L-SIGN transfected cell line.

3. The method of claim 1, wherein said DC-SIGN or L-SIGN is attached to a solid surface.

4. The method of claim 1, wherein said measuring step is by enzyme-linked immunosorbent assay.

5. The method of claim 1, wherein said measuring step includes the following additional steps:
c. permeabilizing transfected cells;
d. exposing said permeabilized cells to anti-dengue virus antibody;
e. detecting the binding of said antibody.

6. The method of claim 1, wherein said detection assay is conducted on a microscope slide.

7. The method of claim 2, wherein said transfected cell is a Raji B cell.

8. The method of claim 3, wherein said solid surface is a microbead.

9. The method of claim 3, wherein said solid surface is a culture dish.

10. The method of claim 5, wherein said detecting step is by immunocytometry.
